(19) United States
(12) Patent Application Publication
(10) Pub. No.: US 2016/0296204 A1
(43) Pub. Date: Oct. 13, 2016

(54) ULTRASONIC IMAGING APPARATUS AND METHOD OF CONTROLLING THE SAME

(71) Applicant: Samsung Electronics Co., Ltd., Gyeonggi-do (KR)

(72) Inventors: Su Hyun Park, Gyeonggi-do (KR); Kyu Hong Kim, Seoul (KR); Bae Hyung Kim, Gyeonggi-do (KR); Jong Keun Song, Gyeonggi-do (KR); Seung Heun Lee, Gyeonggi-do (KR); Kyung IL Cho, Seoul (KR)

(21) Appl. No.: 15/037,656

(22) PCT Filed: Nov. 18, 2014
(86) PCT No.: PCT/KR2014/011071

§ 371 Date: May 18, 2016

(30) Foreign Application Priority Data
Nov. 18, 2013 (KR) 10-2013-0140169

(51) Int. Cl. A61B 8/14; A61B 6/00; G01S 7/52; A61B 8/00

(52) U.S. Cl. CPC A61B 8/14; A61B 8/54; A61B 8/4483; A61B 8/4405; A61B 6/461; G01S 7/5202; G01S 7/52085; G01S 7/52046

(57) ABSTRACT

There are provided an ultrasonic imaging apparatus for imaging an ultrasonic signal and a method of controlling the same. A method of controlling an ultrasonic imaging apparatus according to an embodiment which uses a 2D array probe in which a plurality of elements are two-dimensionally arranged, the method includes setting an ultrasound to be transmitted using all of the plurality of elements and an echo ultrasound to be received using some predetermined elements among the plurality of elements, determining whether a section of interest of an object is included in a weak resolution region determined by the setting, and generating an ultrasound image of the section of interest according to a beamforming method corresponding to focusing of the transmitted ultrasound by transmitting the ultrasound and receiving the echo ultrasound in accordance with the setting when the section of interest is included in the weak resolution region.

### TECHNICAL FIELD

Embodiments of the present invention relate to an ultrasonic imaging apparatus for imaging an ultrasonic signal and a method of controlling the same.

### BACKGROUND ART

An ultrasonic diagnostic apparatus is an apparatus that radiates an ultrasound toward a specific region inside a body from a surface of the body of an object and obtains an image of a section of a soft tissue or blood flow using information on a reflected echo ultrasound in a noninvasive manner.

The ultrasonic diagnostic apparatus is advantageous in that it is small, cheap, can display in real time, and has high safety having no exposure of X-rays. Due to these advantages, the ultrasonic diagnostic apparatus is being widely used for heart, breast, abdomen, urinary organ, and obstetrics diagnoses.

The ultrasonic diagnostic apparatus radiates an ultrasound through an ultrasonic probe and such an ultrasonic probe may be classified by a method of arranging transducer elements. Recently, research on a method in which a 2D array probe having two-dimensionally arranged elements therein is used to radiate an ultrasound and an ultrasound image is generated based thereon has been actively performed.

### DISCLOSURE OF INVENTION

### Technical Problem

The present invention provides an ultrasonic imaging apparatus that can increase a resolution when an ultrasound is transmitted and received using a co-array of a 2D array probe and a method of controlling the same.

### Solution to Problem

According to an aspect of the invention, there is provided a method of controlling an ultrasonic imaging apparatus which uses a 2D array probe in which a plurality of elements are two-dimensionally arranged. The method includes setting an ultrasound to be transmitted using all of the plurality of elements and an echo ultrasound to be received using some predetermined elements among the plurality of elements, determining whether a section of interest of an object is included in a weak resolution region determined by the setting, and generating an ultrasound image of the section of interest according to a beamforming method corresponding to focusing of the transmitted ultrasound by transmitting the ultrasound and receiving the echo ultrasound in accordance with the setting when the section of interest is included in the weak resolution region.

According to another aspect of the invention, there is provided an ultrasonic imaging apparatus. The ultrasonic imaging apparatus includes a control unit configured to set an ultrasound to be transmitted using all of a plurality of elements of a 2D array probe and an echo ultrasound to be received using some predetermined elements among the plurality of elements, a computing unit configured to determine whether a section of interest of an object is included in a weak resolution region determined by the setting, a 2D array probe configured to transmit the ultrasound and receive the echo ultrasound according to the setting, a beamformer configured to generate an echo signal by beamforming according to a beamforming method corresponding to focusing of the echo ultrasound when the section of interest is not included in the weak resolution region, and an image processing unit configured to generate an ultrasound image of the section of interest of the object based on the echo signal.

### Advantageous Effects of Invention

In the ultrasonic imaging apparatus and the method of controlling the same according to the embodiment, it is possible to obtain the ultrasound image of a high resolution using the co-array.

In the ultrasonic imaging apparatus and the method of controlling the same according to another embodiment, it is possible to obtain a high frame rate when the ultrasound is focused and transmitted.

### BRIEF DESCRIPTION OF DRAWINGS

These and/or other aspects of the invention will become apparent and more readily appreciated from the following description of the embodiments, taken in conjunction with the accompanying drawings of which:

FIG. 1 is a perspective view illustrating an ultrasonic imaging apparatus according to an embodiment;

FIGS. 2A to 2C are diagrams illustrating a 2D array probe as an exemplary ultrasonic probe;

FIG. 3 is a diagram illustrating a control block diagram of an ultrasonic imaging apparatus according to an embodiment;

FIGS. 4A and 4B are diagrams illustrating an exemplary co-array;

FIG. 5 is a diagram illustrating a weak resolution region;

FIGS. 6A and 6B are graphs illustrating a point spread function of an echo ultrasound received by a focused ultrasound;

FIG. 7 is a diagram illustrating ultrasound focusing when an ultrasound is transmitted;

FIG. 8 is a diagram illustrating concepts of a focal point, a virtual source, and a virtual aperture;

FIG. 9 is a diagram illustrating an exemplary method of transmitting and receiving an ultrasound according to retrospective transmit beamforming;

FIGS. 10A and 10b are graphs illustrating a point spread function of an echo ultrasound received by a plane wave ultrasound;

FIG. 11 is a diagram illustrating a method of transmitting an ultrasound for coherent angular compounding;

FIGS. 12A to 12C are diagrams illustrating an exemplary ultrasound image obtained by focusing an ultrasound;

FIGS. 13A to 13C illustrate exemplary ultrasound images obtained by transmitting a plane wave ultrasound;

FIG. 14 is a flowchart illustrating a method of controlling an ultrasonic imaging apparatus according to an embodiment;

FIG. 15 is a flowchart illustrating a method of performing retrospective transmit beamforming using a co-array according to an embodiment; and

FIG. 16 is a flowchart illustrating a method of performing coherent angular compounding using a co-array according to an embodiment.

### BEST MODE FOR CARRYING OUT THE INVENTION

Hereinafter, an ultrasonic imaging apparatus and a method of controlling the same according to an embodiment will be described in detail with reference to the accompanying drawing.

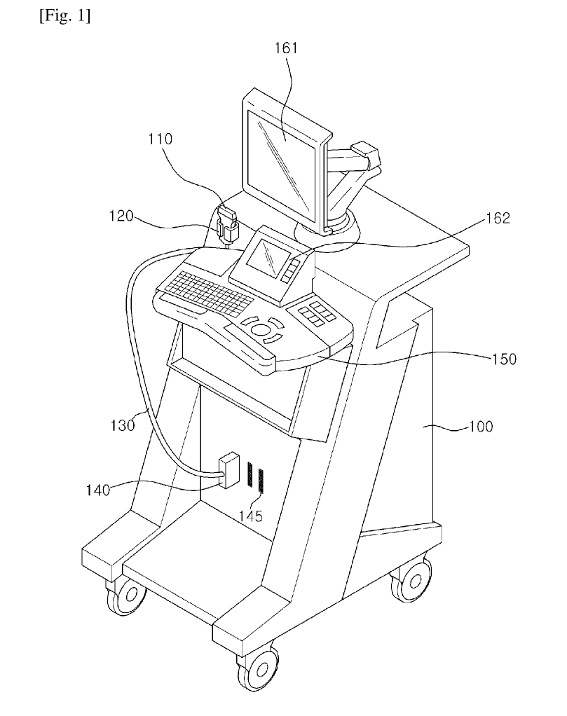

FIG. 1 is a perspective view illustrating an ultrasonic imaging apparatus according to an embodiment. As illustrated in FIG. 1, the ultrasonic imaging apparatus may include a main body 100, an ultrasonic probe 110, an input unit 150, and a display unit 160.

At least one female connector 145 may be provided in a side of the main body 100. A male connector 140 connected to a cable 130 may be physically combined to the female connector 145.

Meanwhile, a plurality of castors (not illustrated) for moving the ultrasonic imaging apparatus may be provided below the main body 100. The plurality of castors enable the ultrasonic imaging apparatus to be fixed at a specific place or to move in a specific direction.

The ultrasonic probe 110 is a unit that comes in contact with a surface of a body of an object and may transmit and receive an ultrasound. Specifically, the ultrasonic probe 110 transmits the ultrasound to an inside of the object according to a transmission signal provided from the main body 100, and receives an echo ultrasound reflected from a specific region inside the object and transmits the echo ultrasound to the main body 100. An end of the cable 130 may be connected to this ultrasonic probe 110 and the male connector 140 may be connected to the other end of the cable 130. The male connector 140 connected to the other end of the cable 130 may be physically combined to the female connector 145 of the main body 100.

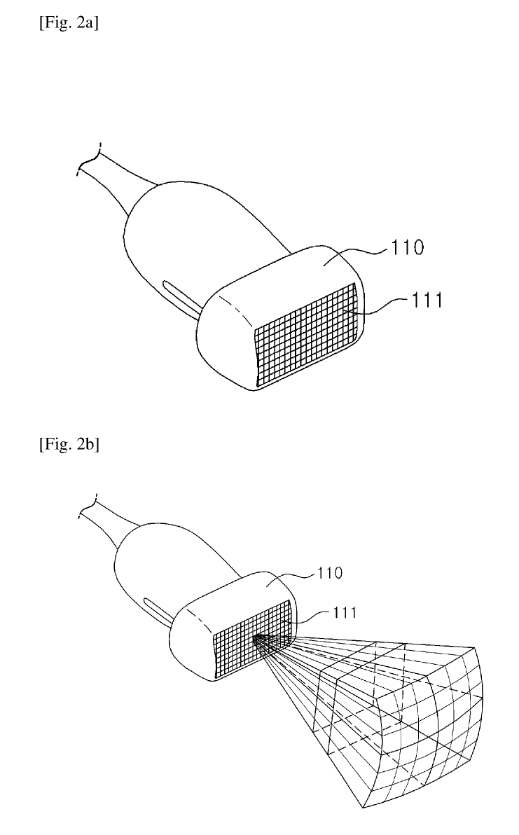

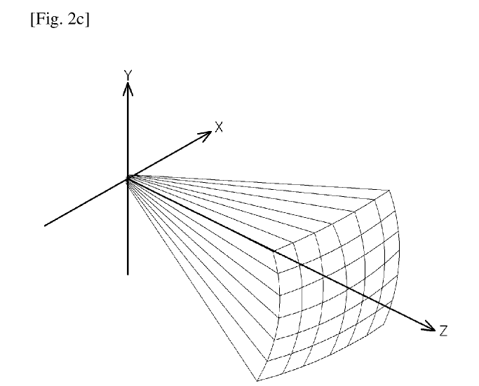

Hereinafter, a 2D array probe as an exemplary ultrasonic probe will be described with reference to FIGS. 2A to 2C. FIG. 2A is a diagram illustrating an appearance of a 2D array probe according to an embodiment. FIG. 2B is a diagram illustrating an exemplary pyramid scan of an ultrasound using a 2D array probe according to an embodiment.

A kind of the ultrasonic probe may be classified according to a method of arranging transducer elements. A 1D array probe in which elements are one-dimensionally arranged in a surface of the ultrasonic probe includes a linear array probe in which elements are arranged in a straight line, a phased array probe, and a convex array probe in which elements are arranged in a curved line. On the other hand, an ultrasonic probe in which elements are two-dimensionally arranged is referred to as a 2D array probe.

As illustrated in FIG. 2A, elements may be two-dimensionally arranged in a surface of a 2D array probe 110. While FIG. 2A exemplifies a case in which elements are arranged on a plane, elements may also form a curved surface and be arranged in the 2D array probe 110.

As illustrated in FIG. 2B, the 2D array probe 110 may transmit the ultrasound to a larger region than the 1D array probe. In particular, when the ultrasound is transmitted using one-dimensionally arranged elements, obtained information may represent a section of the object. However, when the ultrasound is transmitted using two-dimensionally arranged elements, it is possible to obtain information on a volume of the object.

In this way, since an amount of information obtained by the 2D array probe 110 is larger than that of the 1D array probe, hardware complexity increases. Therefore, in order to address this problem, it is possible to use a co-array. This will be described below.

FIG. 2C is a diagram illustrating the ultrasound that is transmitted using a pyramid scan through the 2D array probe 110 in a 3D space. In FIG. 2C, transducer elements in the form of a rectangle are arranged on an xy plane, and it is possible to obtain information on the volume of the object using the ultrasound transmitted from the arranged elements.

Hereinafter, an x axis direction is referred to as a lateral or azimuthal direction, a y axis direction is referred to as an elevational direction, and a z axis direction is referred to as an axial direction.

Referring again to FIG. 1, the input unit 150 is a unit that can receive a command related to an operation of the ultrasonic imaging apparatus. For example, a mode selecting command such as an A-mode (amplitude mode), a B-mode (brightness mode), and an M-mode (motion mode), or an ultrasound diagnosis starting command may be received. The command input through the input unit 150 may be transmitted to the main body 100 via wired and/or wireless communication.

The input unit 150 may include at least one of, for example, a keyboard, a foot switch, and a foot pedal. The keyboard may be implemented in the form of hardware and located above the main body 100. This keyboard may include at least one of a switch, a key, a joystick, and a trackball. As another example, the keyboard may also be implemented in the form of software such as a graphic user interface. In this case, the keyboard may be displayed through a sub-display unit 162 or a main display unit 161. The foot switch or the foot pedal may be provided below the main body 100, and a manipulator may control operations of an ultrasound image generating apparatus using the foot pedal.

The display unit 160 may include the main display unit 161 and the sub-display unit 162.

The sub-display unit 162 may be provided in the main body 100. FIG. 1 illustrates a case in which the sub-display unit 162 is provided above the input unit 150. The sub-display unit 162 may display an application related to an operation of the ultrasound image generating apparatus. The sub-display unit 162 may display, for example, an instruction or a menu necessary for ultrasound diagnosis. This sub-display unit 162 may be implemented as, for example, a cathode ray tube (CRT), or a liquid crystal display (LCD).

The main display unit 161 may be provided in the main body 100. FIG. 1 illustrates a case in which the main display unit 161 is provided above the sub-display unit 162. The main display unit 161 may display an ultrasound image that is obtained in an ultrasound diagnosis process. This main display unit 161 may be implemented as the CRT or the LCD like the sub-display unit 162. FIG. 1 illustrates a case in which the main display unit 161 is combined to the main body 100. However, the main display unit 161 may also be detachable from the main body 100.

FIG. 1 illustrates a case in which both the main display unit 161 and the sub-display unit 162 are provided in the ultrasonic imaging apparatus. However, in some cases, the sub-display unit 162 may not be provided. In this case, the application, the menu, or the like displayed through the sub-display unit 162 may be displayed through the main display unit 161.

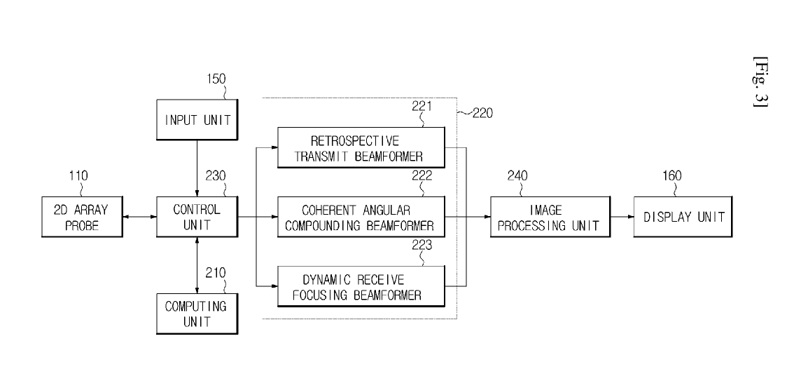

FIG. 3 is a diagram illustrating a control block diagram of an ultrasonic imaging apparatus according to an embodiment.

The ultrasonic imaging apparatus according to the embodiment may include a control unit 230 that sets the ultrasound to be transmitted using all of the plurality of elements of the 2D array probe and the echo ultrasound to be received using some predetermined elements among the plurality of elements, a computing unit 210 configured to determine whether a section of interest of the object is included in a weak resolution region determined by the setting, a 2D array probe configured to transmit the ultrasound and receive the echo ultrasound according to the setting, a beamformer 220 configured to generate an echo signal by beamforming according to a beamforming method corresponding to focusing of the echo ultrasound when the section of interest is included in the weak resolution region, and an image processing unit 240 configured to generate an ultrasound image of the section of interest of the object based on the echo signal. In addition, a display for displaying the generated ultrasound image may be further included.

The ultrasonic imaging apparatus according to the embodiment may use the 2D array probe 110 for transmitting the ultrasound to the object. As illustrated in FIGS. 2A to 2C, the transducer elements are two-dimensionally arranged in the surface of the 2D array probe 110 and thus it is possible to obtain volume data on the object.

Since the 2D array probe 110 has the plurality of elements involved in transmission and reception of the ultrasound, there is a problem of hardware complexity. In particular, when the ultrasound is transmitted to the object using all elements of the 2D array probe 110 and its corresponding echo ultrasound is received using all elements, an amount of information to be processed in the following beamforming and image processing procedures and resulting computational complexity significantly increase.

In order to address such problems, the 2D array probe 110 may utilizes the co-array. Here, the term “co-array” refers to an array scheme that gives an effect of transmission and reception of the ultrasound by a combination of a transmit aperture and a receive aperture.

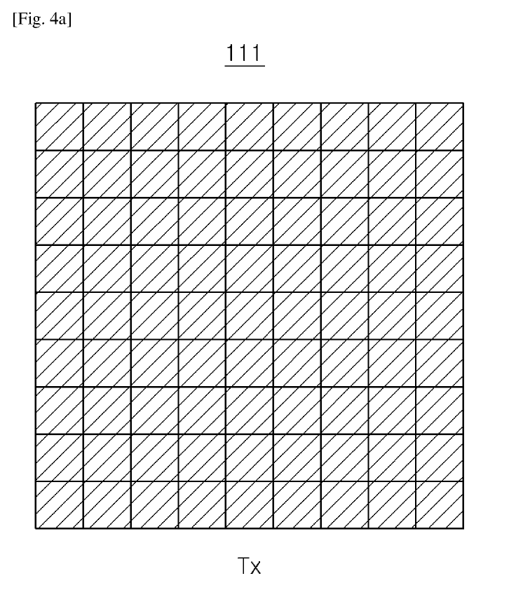

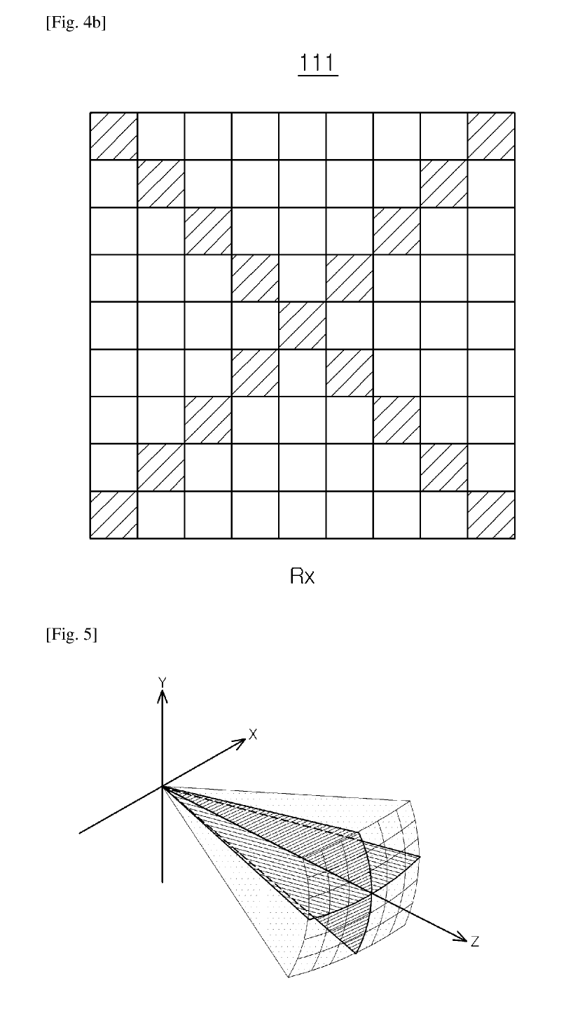

FIGS. 4A and 4B are diagrams illustrating an exemplary co-array. Shaded portions indicate actually used elements.

FIG. 4A illustrates exemplary elements used when the 2D array probe transmits the ultrasound. FIG. 4A exemplifies a case in which all of the plurality of elements of the 2D array probe are used to transmit the ultrasound. As mentioned above, the 2D array probe 110 may transmit the ultrasound using the two-dimensionally arranged elements. This may generate the echo ultrasound for a wider range than that of the 1D array probe without moving the probe itself.

FIG. 4B illustrates exemplary elements used when the 2D array probe 110 receives the echo ultrasound. When the echo ultrasound generated in a wide range is received in all elements of the 2D array probe 110, resulting hardware complexity and the amount of information to be processed increase. In order to address these problems, it is possible to receive the echo ultrasound using only some of the plurality of elements of the 2D array probe 110. In FIG. 4B, an X-shape array is used to receive the echo ultrasound.

When the echo ultrasound is received using some elements of the 2D array probe 110, an amount of obtained information may be smaller than that of a case in which the echo ultrasound is received using all elements. In this case, there is a concern about resolution degradation of the generated ultrasound image. This will be described along with the computing unit 210 to be described.

While FIGS. 4A and 4B illustrate exemplary co-arrays, it is possible to set an element used for transmission and an element used for reception as necessary. When a user inputs a desired co-array through the input unit, the control unit 230 may set the elements based on the user's input, or the control unit 230 may set the elements based on an internal computation result of the apparatus or hardware implementation.

Hereinafter, for convenience of description, it is assumed that the co-array used by the 2D array probe 110 uses all elements for transmission and uses the X-shape array for reception. However, this is only an example of the ultrasonic imaging apparatus and the method of controlling the same, and the invention is not limited thereto.

The computing unit 210 may determine whether the section of interest of the object is included in the weak resolution region determined by co-array setting of the 2D array probe 110. Here, the term “weak resolution region” refers to a region in which transmission and reception of the ultrasound using all elements of the 2D array probe 110 are not equivalent to transmission and reception of the ultrasound using the co-array. The term “section of interest” of the object refers to a position inside the object of which the ultrasound image is finally generated and may be determined by an input by the user through the input unit or internal computation of the apparatus.

When the echo ultrasound is received using only some elements rather than echo ultrasound reception using all elements, there is a risk of obtaining a small amount of information in a specific region. Since this causes resolution degradation of a finally generated ultrasound image, this region becomes the weak resolution region. When the echo ultrasound is received in the weak resolution region, it is possible to prevent resolution degradation of the generated ultrasound image by performing corresponding beamforming.

The weak resolution region may be determined by co-array setting.

Mathematically, the co-array is defined as a set of vector sums of positions of a transmission element and a reception element. A set C of a co-array pair of a transmit aperture and a receive aperture is defined as Equation 1.

C={y|y=x₁+x₂, for x₁ ∈ A_{T} and x₂ ∈ A_{R}} Equation 1

Here, AT represents a set of points at the transmit aperture and AR represents a set of points at the receive aperture.

Co-array computation may be represented as convolution of the transmit aperture and the receive aperture. Equation 2 represents co-array computation when all elements are used to transmit the ultrasound and all elements are used to receive the echo ultrasound in the 2D array probe 110 of a size of N×M. Equation 3 represents co-array computation when all elements are used to transmit the ultrasound and the X-shape array is used to receive the echo ultrasound in the 2D array probe 110 of a size of N×M.

$$\begin{matrix}{H}_{FT - FR} ( α , β ) ∝ ( \frac{sin ( α N )}{sin ( α )} \frac{sin ( β M )}{sin ( β )} ) ( \frac{sin ( α N )}{sin ( α )} \frac{sin ( β M )}{sin ( β )} ) & Equation 2 \\ {H}_{FT - XR} ( α , β ) ∝ ( \frac{sin ( α N )}{sin ( α )} \frac{sin ( β M )}{sin ( β )} ) ( \frac{sin ( ( α - β ) N )}{sin ( α - β )} + \frac{sin ( ( α + β ) M )}{sin ( α - β )} ) & Equation 3\end{matrix}$$

Here, λ represents a wavelength of a transmitted ultrasound, d represents a pitch of elements, and α and β satisfy Equation 4 and Equation 5.

$$\begin{matrix}α = \frac{π dx}{λ z} & Equation 4 \\ β = \frac{π dy}{λ z} & Equation 5\end{matrix}$$

Here, x, y, and z represent coordinates in the space described in FIG. 2C.

Equations 2 and 3 represent a Fourier transform relation between a discrete aperture space and a continuous image space (α, β) in the 2D array probe 110 of a size of N×M.

When α or β is set to 0, Equations 2 and 3 are the same. When α is 0, it refers to a yz plane, and when β is 0, it refers to an xz plane. Therefore, when the ultrasound is transmitted to the xz plane or the yz plane and the echo ultrasound is received, it can be verified that reception of the echo ultrasound using all elements and reception of the echo ultrasound using the X-shape array have the same result.

However, when the ultrasound is transmitted to an xy-z plane and the echo ultrasound is received, α and β have a real value other than 0. When these α and β are assigned to Equations 2 and 3, results thereof become different to each other. Accordingly, reception of the ultrasound using all elements and reception of the echo ultrasound using the X-shape array may no longer equivalent.

When an echo ultrasound reception result is not equivalent to a case of using all elements, it may cause resolution degradation of the generated ultrasound image. When ultrasound diagnosis is performed based on such an image, accuracy decreases and heath of a patient may be threatened.

In order to address this problem, a set of planes in which reception of the echo ultrasound using all elements and reception of the echo ultrasound using the co-array are not equivalent is set as the weak resolution region, and when the echo ultrasound is collected using the co-array for this region, it is possible to perform appropriate beamforming on the collected echo ultrasound. In this case, the beamforming to be performed may include an additional process for preventing resolution degradation of the ultrasound image in addition to general dynamic receive focusing.

Equation 3 represents a case in which the echo ultrasound is received using the X-shape array, but this is only an example, and related Equation may differ according to a shape of the co-array. Accordingly, when the co-array to be used is set, Equation corresponding to the setting is determined, and the weak resolution region may be determined according to such Equation.

The computing unit 210 may determine whether the section of interest of the object selected by the user's input or internal computation of the apparatus is included in the weak resolution region determined in this way.

As illustrated in FIG. 5, slashed regions in a 3D space refer to an xz plane and a yz plane. In these regions, even when the echo ultrasound is received using the X-shape array, it is equivalent to a case of using all elements. However, in a shaded region other than the slashed regions, when the X-shape array is used, a different result from a case of using all elements is obtained. Therefore, in this case, the shaded region is determined as the weak resolution region, and the computing unit 210 determines whether a section of interest of the object is included in the weak resolution region.

When the computing unit 210 determines that the section of interest is included in the weak resolution region, the beamformer 220 may perform beamforming according to a beamforming method corresponding to focusing of the transmitted ultrasound and generate an echo signal.

As mentioned above, the beamformer 220 may perform the beamforming by adding an additional process for preventing resolution degradation generated when the co-array is used. Exemplary beamforming performed in the beamformer 220 may include dynamic receive focusing, retrospective transmit beamforming, or coherent angular compounding.

Examples of the beamformer 220 may include a dynamic receive focusing beamformer 223 for performing dynamic receive focusing, a retrospective transmit beamformer 221 for performing retrospective transmit beamforming, or a coherent angular compounding beamformer 222.

A method of beamforming performed by the beamformer 220 will be specifically described along with the control unit 230.

The control unit 230 may set the co-array of the 2D array probe 110. As mentioned above, it is possible to set such that all of the plurality of elements are used for transmission and the X-shape array is used for reception.

Also, the control unit 230 may control a steering scheme of the ultrasound according to focusing of the ultrasound to be transmitted. This is because focusing of the ultrasound to be transmitted determines a beamforming method to be performed by the beamformer 220 later.

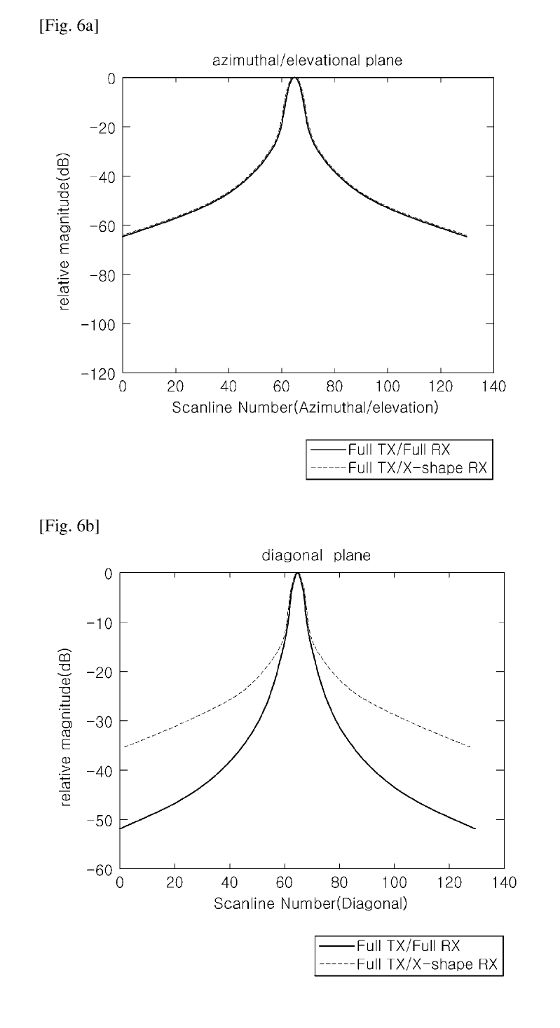

Even when the transmitted ultrasound is focused, the weak resolution region is determined by co-array setting. When the section of interest is included in the weak resolution region, it is difficult to generate an accurate ultrasound image. This may be experimentally verified through FIGS. 6A and 6B.

FIGS. 6A and 6B are graphs illustrating a point spread function of an echo ultrasound received by a focused ultrasound. In particular, in FIG. 6A, the section of interest is an xz plane or a yz plane, and in FIG. 6B, the section of interest is an xz plane or a yz plane that is rotated at 45° around a z axis. The section of interest in FIG. 6B is referred to as a diagonal plane.

In FIGS. 6A and 6B, a solid line indicates a point spread function of the echo ultrasound received when the ultrasound is transmitted using all elements and the echo ultrasound is received using all elements, and a dotted line indicates a point spread function of the echo ultrasound received when the ultrasound is transmitted using all elements and the echo ultrasound is received using the X-shape array.

As illustrated in FIG. 6A, when the focused ultrasound is transmitted and the echo ultrasound generated from the xz plane or the yz plane is received, reception using all elements and reception using the co-array have an equivalent result. Therefore, in this case, it is possible to perform general beamforming. Here, the general beamforming may refer to beamforming according to fixed transmit focusing and dynamic receive focusing methods.

However, as in FIG. 6B, when the focused ultrasound is transmitted and the echo ultrasound generated from the diagonal plane is received, reception using all elements and reception using the co-array may have different results.

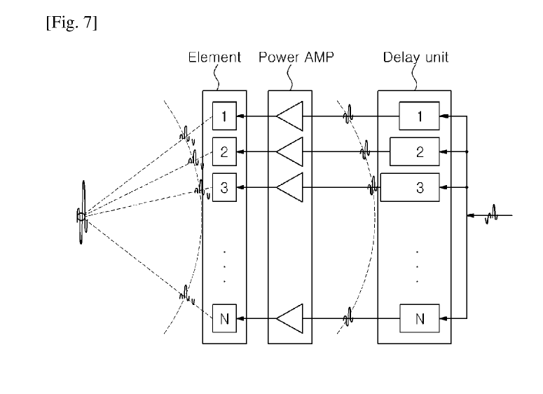

In order to address this problem, it is possible to use a retrospective transmit beamforming method. Hereinafter, the retrospective transmit beamforming method will be described with reference to FIGS. 7 to 9.

FIG. 7 is a diagram illustrating ultrasound focusing when the ultrasound is transmitted.

In consideration of a different distance from a focal point that is a position at which the ultrasound is focused to each element, a different time delay is assigned and thus a transmission signal at only the focal point may be maximized. Specifically, a transmission signal for generating the ultrasound is generated. The transmission signal is delivered to a transmission delay unit (delay unit), and the transmission delay unit may apply a different time delay to the received transmission signal. The transmission signal to which the time delay is applied may be delivered to the plurality of transducer elements through a power amp. Through this process, the ultrasound output from the transducer has the same phase when it arrives at the focal point based on the different transmission time delay.

Dynamic transmit focusing refers to that an ultrasonic signal is focused at a plurality of focal points positioned in a single scanline multiple times. For example, when ultrasonic signals are focused at 10 focal points positioned in any of the plurality of scanlines ten times, resolution of the ultrasound image may increase. However, in consideration of a propagation speed (1540 m/s) of the ultrasound delivered inside the object, when transmission is performed, focusing of the ultrasound at 10 focal points positioned in a single scanline may be an obstacle of real time imaging.

The control unit 230 controls the 2D array probe 110 such that the ultrasound is focused at different focal points positioned in different scanlines, and the ultrasound image is obtained by assuming the echo ultrasound reflected from the different focal points as a virtual source, which allows dynamic transmit focusing. In this manner, a method of obtaining the ultrasound image through a plurality of virtual sources may be a synthetic aperture imaging method. In particular, a retrospective transmit beamforming method may be a method of configuring the virtual source such that the virtual source is positioned at a front of the 2D array probe 110 and a spherical wave is propagated to the front and a rear of the virtual source.

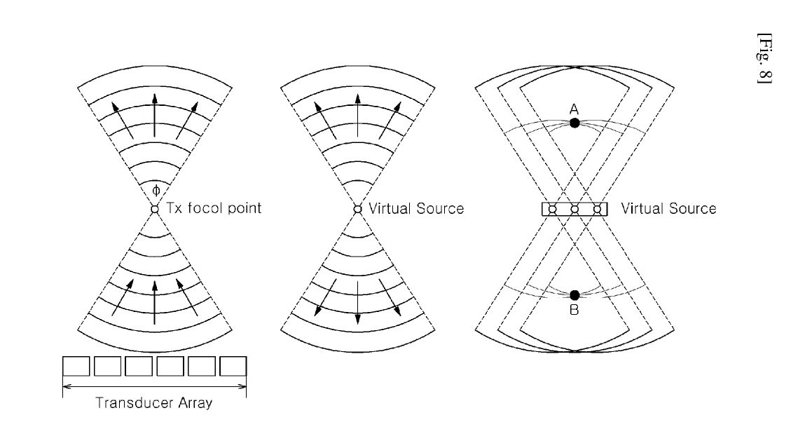

In order to describe the retrospective transmit beamforming method, concepts of a focal point, a virtual source, and a virtual aperture will be described first with reference to FIG. 8.

As illustrated in the left diagram of FIG. 8, the ultrasound generated from a plurality of elements constituting a transducer array is focused at the focal point. A width of the transmitted ultrasound gradually decreases from a transducer to the focal point, and the width of the ultrasound gradually increases after it arrives at the focal point.

It is possible to assume that the virtual source is present at a position of the focal point and the ultrasound is generated from the virtual source. That is, the left diagram of FIG. 8 may be replaced with the middle diagram. As a result, the focal point may be replaced with the virtual source, and it is possible configure a single virtual source by one time of ultrasound transmission.

In general, the echo ultrasound received from the object has information on all regions of the object at which the ultrasound transmitted from the plurality of elements arrive. When the width of the transmitted ultrasound is large, it is possible to obtain information on a larger region. Therefore, when the ultrasound is steered and transmitted, that is, when the ultrasound is transmitted along the plurality of scanlines, the ultrasound propagating along any scanline may include information on an image point in another scanline.

As illustrated in the right diagram of FIG. 8, there are three virtual sources and these form the virtual aperture. A transmission region generated in each virtual source includes information corresponding to both image points A and B. The ultrasound transmitted from the virtual aperture may arrive at the image points A and B with different time delays, and when the dynamic receive focusing is performed, it is possible to perform the dynamic transmit focusing through additional variable delay compensation for transmission. Hereinafter, the retrospective transmit beamforming method as one of the dynamic transmit focusing methods will be described.

FIG. 9 is a diagram illustrating an exemplary method of transmitting and receiving the ultrasound according to retrospective transmit beamforming on the assumption of a linear scan.

A plurality of elements included in a group A among the plurality of elements of the 2D array probe 110 may transmit the ultrasound toward a first focal point f0 positioned in a first scanline L0 among the plurality of scanlines. In this case, different delay times may be applied to the plurality of ultrasounds transmitted from each element included in the group A. In this way, the plurality of ultrasounds may be focused at the first focal point f0. An element 0 that receives the echo ultrasound among the plurality of elements may receive a first echo ultrasound generated by the ultrasound transmitted from the element included in the group A to the first focal point f0.

In the same manner, elements included in a group B among the plurality of elements may transmit the ultrasound toward a second focal point f2 positioned in a second scanline L2 among the plurality of scanlines. Also, different delay times may be applied to the plurality of ultrasounds transmitted from each element included in the group B. In this way, the plurality of ultrasounds may be focused at the second focal point f2. An element 0 among the plurality of elements may receive a second echo ultrasound generated by the ultrasound transmitted from the element included in the group B to the second focal point f2.

Each of P1, P2, P3, and P4 of FIG. 9 refers to an image point. These image points are included in each of the plurality of scanlines defined from the plurality of elements. For example, P1 and P2 are image points included in the first scanline L0. Meanwhile, since P1′ and P1 are in the same concentric circle (dotted line) around f0, an arriving time of the ultrasound from P1′ to the first focal point f0 positioned in the first scanline L0 and an arriving time of the ultrasound from P1 to the first focal point f0 are the same.

The first echo ultrasound and the second echo ultrasound which are received by the reception element may include information on the image point P1. Specifically, the image point P1 is one of the plurality of image points included in the first scanline L0, the first echo ultrasound may include a component reflected from the image point P1, and the second echo ultrasound may also include the component reflected from the image point P1.

For example, an ultrasound transmitted from the element 0 included in the group A toward the first focal point f0 of the first scanline L0 propagates through a path of Z1 for a time t1 and arrives at the image point P1. As a result, an echo ultrasound reflected at the image point P1 may propagate through the path of Z1 for the time t1 and be received in the element 0.

Also, an ultrasound transmitted from an element 2 included in the group B toward the second focal point f2 of the second scanline L2 propagates through a path of Z2 for a time t2 and arrives at the image point P1. An echo ultrasound reflected at the image point P1 may propagate through the path of Z1 for a time t3 and be received in the element 0. In this case, an ultrasound transmitted toward the second focal point f2 along the second scanline may propagate through the path of Z2 for the time t2 from the element 2. Since a position thereof and P1 are in the same concentric circle (dotted line) around f2, a time t3 necessary for the echo ultrasound to arrive at a reception transducer 0 from the position and a time t1 necessary for the echo ultrasound to arrive at the reception transducer 0 from the image point P1 are the same.

It is possible to generate an echo signal by beamforming the first echo signal and the second echo signal, which are received in the reception element. For example, an appropriate reception delay time is applied to the second echo ultrasound and the second echo ultrasound to which the reception delay time is applied and the first echo ultrasound may be synthesized. In addition, a third echo ultrasound is further received and thus it is also possible to synthesize the first echo ultrasound, the second echo ultrasound, and the third echo ultrasound. In this case, the third echo ultrasound may refer to an echo ultrasound received in the element 0 after the ultrasound is transmitted to a different focal point positioned in a different scanline from a plurality of elements included in a different group.

It is possible to adjust the reception delay time applied to the second echo ultrasound such that echo ultrasounds reflected at each of the plurality of image points positioned in the scanline of the reception element are added at the same time. For example, in order to increase a phase of the echo ultrasound reflected at the image point P1, an appropriate reception time delay is applied to the second echo ultrasound reflected at the image point P1 and the second echo ultrasound and the first echo ultrasound are synthesized. This synthesis is called a coherent sum.

When the plurality of echo ultrasounds received in the reception element are added, it is possible to generate an echo signal that is a basis of the ultrasound image.

When the retrospective transmit beamforming is performed in this way, it is possible to perform the dynamic transmit focusing in each of the image points existing in the scanline defined from the reception element using the virtual sources included in the virtual aperture.

Although the retrospective transmit beamforming has been described on the assumption of the linear scan in FIG. 9, it is possible to perform the retrospective transmit beamforming using the same method even when the focused ultrasound is steered (a pyramid scan).

In order to perform the retrospective transmit beamforming described in FIGS. 7, 8, and 9, the control unit 230 may control the 2D array probe 110. Before this control, it is assumed that the control unit 230 performs co-array setting of the 2D array probe 110.

Specifically, the control unit 230 may control the 2D array probe 110 such that the plurality of ultrasounds are radiated onto the plurality of focal points inside the object along the plurality of scanlines using all of the plurality of elements. In addition, it is possible to control the 2D array probe 110 such that the plurality of echo ultrasounds including information on the inside of the object in which the plurality of scanlines are positioned are received using some of the plurality of elements, for example, the X-shape array. That is, it is possible to steer and transmit the ultrasound to the plurality of focal points using the co-array.

According to this control of the control unit 230, the 2D array probe 110 may receive the plurality of echo ultrasounds.

The retrospective transmit beamformer 221 may perform a coherent sum of at least two echo ultrasounds that include information on the same position inside the object among the plurality of echo ultrasounds received in this way. Based on a result of the coherent sum, it is possible to generate each echo signal corresponding to each scanline When the retrospective transmit beamforming method that assumes the plurality of virtual sources is used, transmission focusing is performed at the plurality of image points. As a result, it is possible to address a problem in which the resolution of the ultrasound image decreases in a region other than the focal point.

Referring again to FIG. 3, even when the plane wave ultrasound is transmitted without focusing, the control unit 230 may control the steering scheme of the ultrasound.

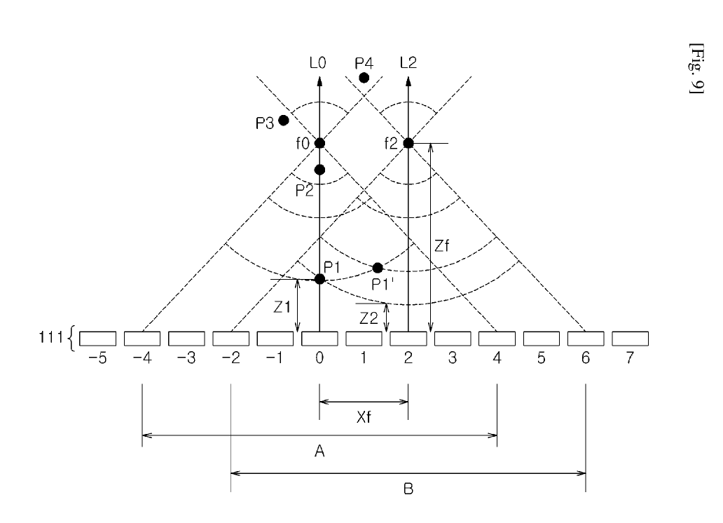

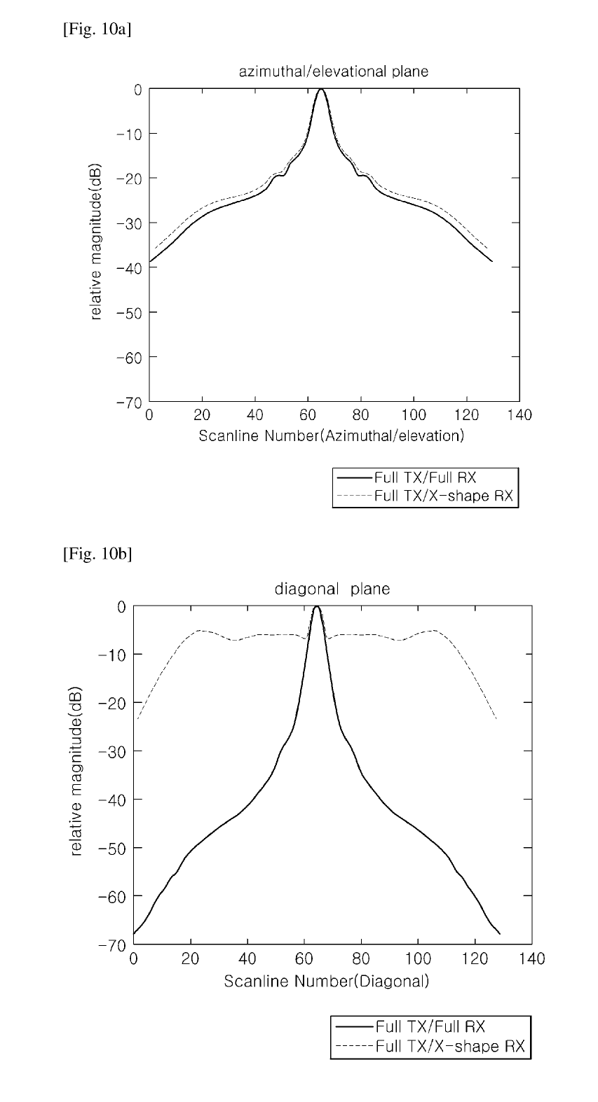

Even when the ultrasound is transmitted without focusing, the problem of resolution degradation due to the co-array setting may occur. Hereinafter, this problem will be described through a simulation result with reference to FIGS. 10A and 10B.

FIGS. 10A and 10B are graphs illustrating a point spread function of the echo ultrasound received by the plane wave ultrasound. In particular, in FIG. 10A, the section of interest is an xz plane or a yz plane, and in FIG. 10B, the section of interest is an xz plane or a yz plane that is rotated at 45° around a z axis. The section of interest in FIG. 10B is referred to as the diagonal plane.

In FIGS. 10A and 10B, a solid line indicates a point spread function of the echo ultrasound received when the ultrasound is transmitted using all elements and the echo ultrasound is received using all elements, and a dotted line indicates a point spread function of the echo ultrasound received when the ultrasound is transmitted using all elements and the echo ultrasound is received using the X-shape array.

As illustrated in FIG. 10B, when an unfocused plane wave ultrasound is transmitted and the echo ultrasound generated from the xz plane or the yz plane is received, reception using the all elements and reception using the co-array show similar results. Therefore, in this case, it is possible to perform general beamforming. Here, the general beamforming may refer to a process of receiving a plane wave echo ultrasound and generating the echo signal through the dynamic receive focusing.

However, as illustrated in FIG. 10B, when the plane wave ultrasound is transmitted and the echo ultrasound generated from the diagonal plane is received, it may be verified that reception using the all elements and reception using the co-array show significantly different results.

In order to address this problem, it is possible to use coherent angular compounding.

The coherent angular compounding refers to that the plane wave ultrasound is transmitted in various angles when transmission is performed, its corresponding plane wave echo ultrasound is received, and then the echo signal is generated by synthesizing the received ultrasounds. Since, in the coherent angular compounding, the echo signal for the ultrasound image is generated by transmitting and receiving the plane wave multiple times, as the number of times of synthesizing is increased, quality and reliability of the generated image may increase.

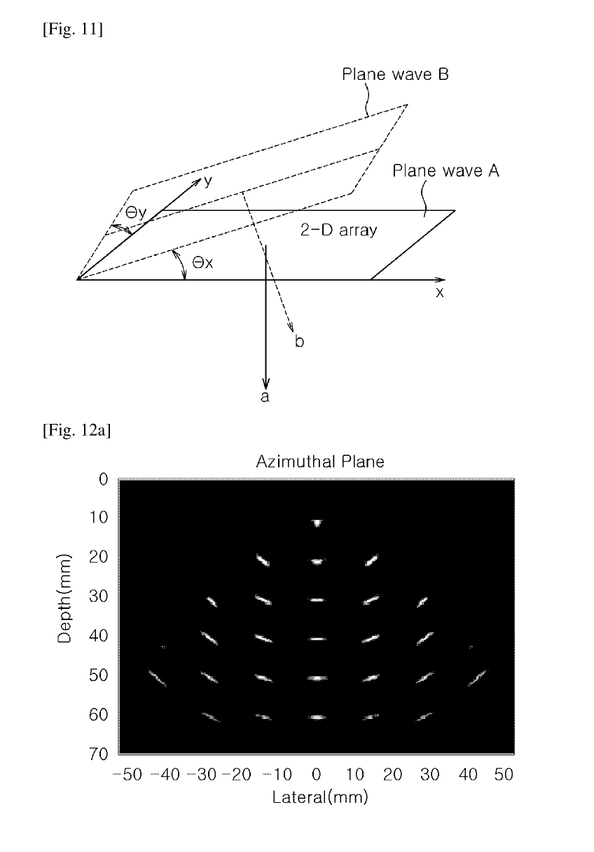

FIG. 11 is a diagram illustrating a method of transmitting the ultrasound for the coherent angular compounding.

The control unit 230 may control the 2D array probe 110 such that the plane wave ultrasound is steered and radiated onto the object for the coherent angular compounding. For this purpose, it is possible to apply a transmission time delay to each element.

As illustrated in FIG. 11, the control unit 230 may control the 2D array probe 110 such that a plane wave A propagating in a direction a is transmitted using all elements. Also, the control unit 230 may control the 2D array probe 110 such that a plane wave B propagating in a direction b is transmitted using all elements. In this manner, the plane waves having different propagating directions are transmitted to the object, and thus it is possible to receive the echo ultrasound for the coherent angular compounding.

In order to steer and transmit the plane wave ultrasound, the control unit 230 may apply the transmission delay time to each element, and this follows Equation 6.

T_{d}=sin θₓ+y sin θ_{y} Equation 6

Here, Td represents the transmission delay time applied to each element, (x,y) represents a position of each element, and Θx or Θy represents a tilt angle with respect to an x axis or a y axis.

The control unit 230 may control the 2D array probe 110 such that the plane wave is steered and transmitted based on the above transmission delay time, and a plurality of corresponding plane wave echo ultrasounds are received using some elements, for example, the X-shape array.

The coherent angular compounding beamformer 222 may perform the coherent sum of at least two echo ultrasounds that include information on the same position among the plurality of echo ultrasounds. Accordingly, the coherent angular compounding beamformer 222 may generate the echo signal that is a basis of the ultrasound image.

Referring again to FIG. 3, the image processing unit 240 may receive the echo signal from the beamformer and convert the received echo signal into the ultrasound image. In this case, the generated ultrasound image may be an image of the section of interest inside the object. Since a method of converting the echo signal into the ultrasound image is well-known to those skilled in the art, detailed description thereof is omitted.

The display unit 160 may display the ultrasound image generated in the image processing unit 240 on a screen.

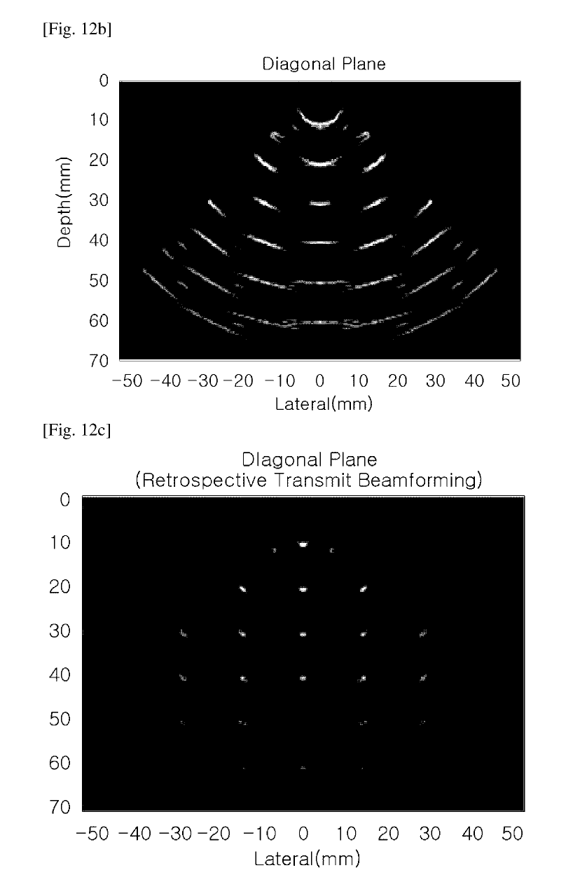

FIGS. 12A to 12C illustrate an exemplary ultrasound image obtained by focusing the ultrasound.

FIG. 12A exemplifies a case in which the section of interest is an xz plane. FIG. 12B exemplifies a case in which the section of interest is a diagonal plane. FIG. 12C exemplifies an image obtained by the retrospective transmit beamforming method when the section of interest is the diagonal plane.

As illustrated in FIG. 12A, when the section of interest is the xz plane, the ultrasound image obtained by transmitting the focused ultrasound has a high lateral resolution. On the other hand, as illustrated in FIG. 12B, when the section of interest is the diagonal plane, it may be verified that a lateral resolution of the ultrasound image decreases. In particular, a resolution of a region other than near 30 mm that is a focus depth of the transmitted ultrasound decreases. In order to improve this problem, bidirectional focusing may be performed through the retrospective transmit beamforming. As a result, it is possible to obtain the ultrasound image of a high lateral resolution as illustrated in FIG. 12C.

The display unit 160 may display the ultrasound image obtained by focusing the ultrasound and the ultrasound image obtained by transmitting the plane wave ultrasound on the screen.

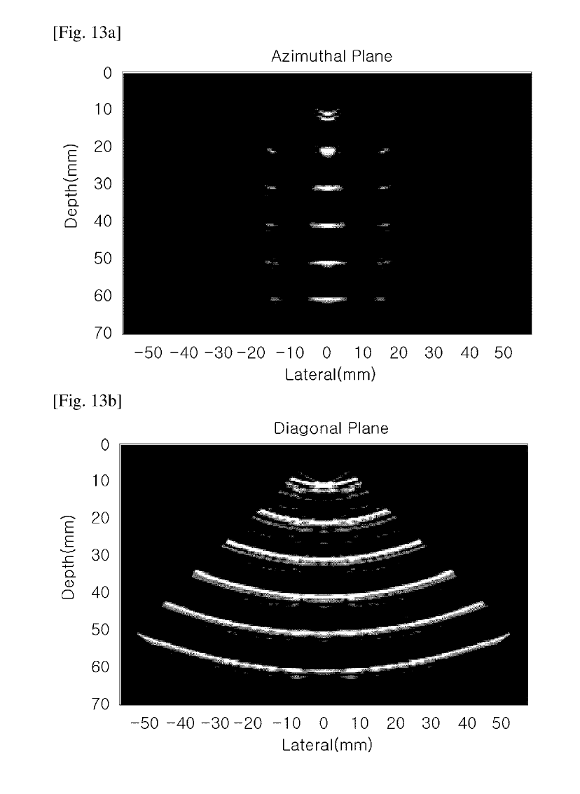

FIGS. 13A to 13C illustrate exemplary ultrasound images obtained by transmitting the plane wave ultrasound.

FIG. 13A exemplifies a case in which the section of interest is an xz plane. FIG. 13B exemplifies a case in which the section of interest is a diagonal plane. FIG. 13C exemplifies an image obtained by the coherent angular compounding when the section of interest is the diagonal plane.

As illustrated in FIG. 13A, when the section of interest is the xz plane, the ultrasound image obtained by transmitting the plane wave ultrasound has a high lateral resolution. On the other hand, as illustrated in FIG. 13B, when the section of interest is the diagonal plane, it may be verified that a lateral resolution of the ultrasound image decreases. In order to improve this problem, the plurality of plane waves having different propagating directions are transmitted, corresponding echo ultrasounds are received, the coherent angular compounding are performed on the received echo ultrasounds, and thus it is possible to obtain the ultrasound image of a high lateral resolution as illustrated in FIG. 13C.

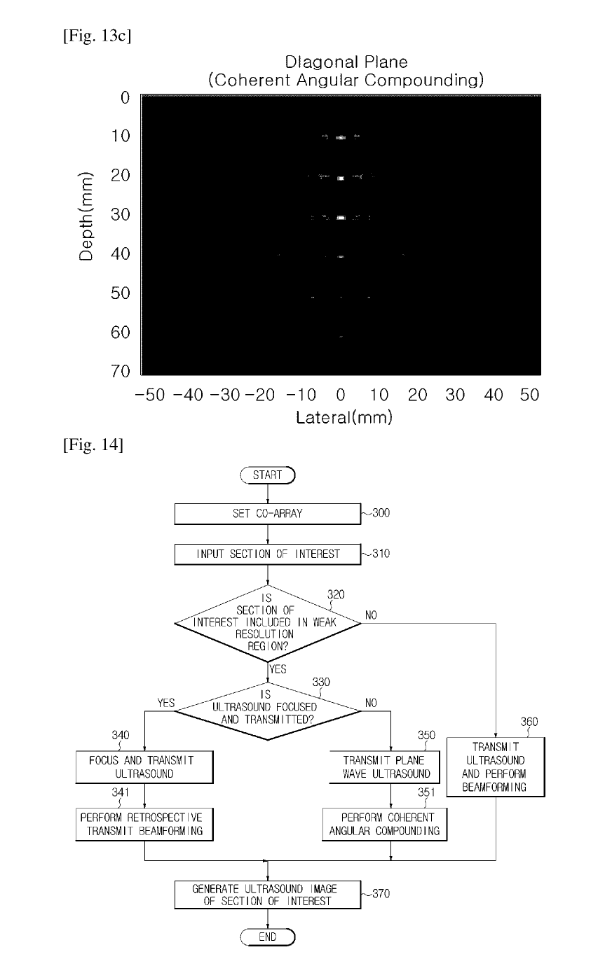

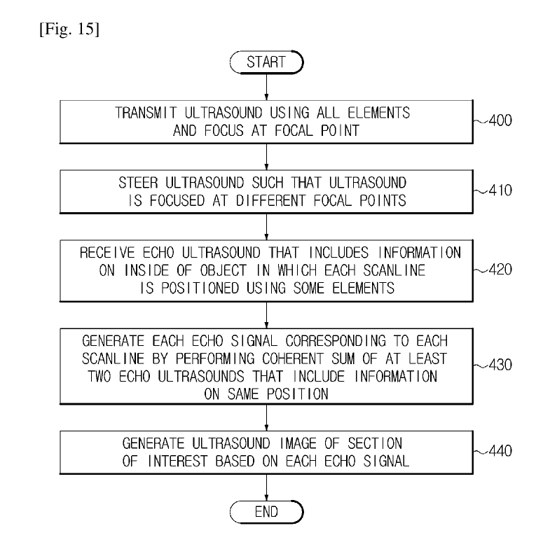

FIG. 14 is a flowchart illustrating a method of controlling an ultrasonic imaging apparatus according to an embodiment.

First, the co-array of the 2D array probe 110 may be set (300). The control unit 230 sets the co-array that is determined by the user or internal computation of the apparatus. In this case, the set co-array may transmit the ultrasound using all of the plurality of elements and receive the echo ultrasound using some of the plurality of elements, for example, the X-shape array.

When the co-array is set, the section of interest inside the object to be generated as the ultrasound image may be input (310). In this case, the section of interest may be input by the user or internal computation of the apparatus.

Next, it is determined whether the input section of interest is included in the weak resolution region (320). The weak resolution region refers to a region in which transmission and reception of the ultrasound using all of the plurality of elements and transmission and reception of the ultrasound using the co-array have different results. In this case, since the resolution of the ultrasound image decreases, it is necessary to perform appropriate beamforming therefor.

When the section of interest is not included in the weak resolution region, the beamforming is performed by transmitting and receiving the ultrasound using a general method (360). Here, the general method may refer to the dynamic receive focusing used when the ultrasound is transmitted and received using all of the plurality of elements. As a result of the beamforming, it is possible to obtain the echo signal.

On the other hand, when the section of interest is included in the weak resolution region, it is necessary to perform appropriate beamforming therefor. Since the beamforming method is determined by focusing of the ultrasound, it is determined first whether the ultrasound is focused and transmitted.

When the ultrasound is focused, the ultrasound is focused and transmitted to the inside of the object (340). In particular, for the retrospective transmit beamforming to be performed, the ultrasound may be steered and transmitted to the plurality of focal points.

After the echo ultrasound is obtained in correspondence with the transmitted ultrasound, the retrospective transmit beamforming is performed based on the obtained echo ultrasound (341). As a result of the retrospective transmit beamforming, it is possible to generate the echo signal. The retrospective transmit beamforming method will be described with reference to FIG. 15.

When the ultrasound is not focused, the plane wave ultrasound may be transmitted to the object (350). In particular, for the coherent angular compounding to be performed, the plurality of plane waves having different propagating directions may be transmitted to the object.

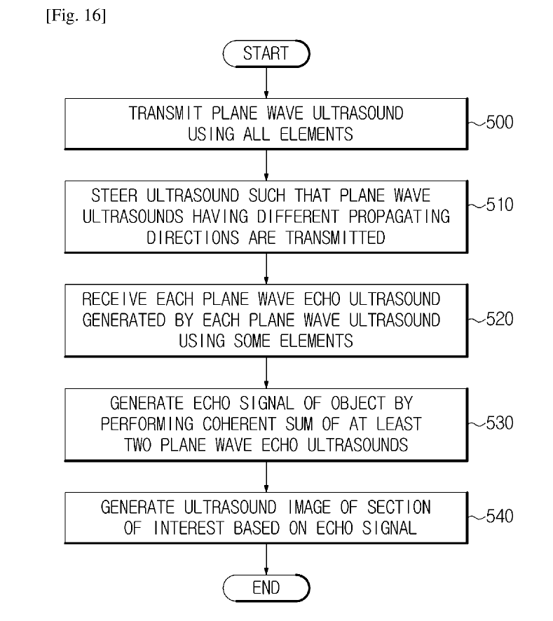

After the echo ultrasound is obtained in correspondence with the transmitted ultrasound, the coherent angular compounding is performed based on the obtained echo ultrasound (351). It is possible to generate the echo signal through the coherent angular compounding. The coherent angular compounding will be described with reference to FIG. 16.

The ultrasound image of the section of interest is generated based on the echo signal generated by the beamforming (370).

FIG. 15 is a flowchart illustrating a method of performing retrospective transmit beamforming using a co-array according to an embodiment.

First, the ultrasound is transmitted using all elements of the 2D array probe and is focused at the focal point inside the object (400). Also, for the beamforming to be performed, the ultrasound may be steered such that the ultrasound is focused at different focal points (410).

Next, the echo ultrasound including information on the inside of the object in which each scanline is positioned may be received using some elements, for example, the X-shape array (420). In this case, when the received echo ultrasound is imaged through the general beamforming, a region other than the focal point has a low resolution.

Accordingly, in order to complement this problem, the retrospective transmit beamforming is performed. That is, the coherent sum of at least two echo ultrasounds that include information on the same position is performed and each echo signal corresponding to each scanline is generated (430). Based on the echo signal generated in this way, it is possible to generate the ultrasound image of the section of interest (440).

FIG. 16 is a flowchart illustrating a method of performing coherent angular compounding using a co-array according to an embodiment.

First, the plane wave ultrasound is transmitted to the object using all elements (500). Also, for the beamforming to be performed, the ultrasound is steered such that the plane wave ultrasounds having different propagating directions are transmitted (510). For this purpose, it is possible to apply the transmission delay time to each element.

Next, each plane wave echo ultrasound generated by each plane wave ultrasound is received using some elements (520). When this is imaged using the general beamforming method, a lateral resolution of the ultrasound image may be significantly low.

In order to complement this problem, the coherent angular compounding may be performed. That is, it is possible to generate the echo signal of the object by performing the coherent sum of at least two plane wave echo ultrasounds (530). Based on the echo signal generated in this way, it is possible to generate the ultrasound image of the section of interest (540).

Although a few embodiments of the present invention have been shown and described, it would be appreciated by those skilled in the art that changes may be made in these embodiments without departing from the principles and spirit of the invention, the scope of which is defined in the claims and their equivalents.

1. A method of controlling an ultrasonic imaging apparatus which uses a 2D array probe in which a plurality of elements are two-dimensionally arranged, comprising:
setting an ultrasound to be transmitted using all of the plurality of elements and an echo ultrasound to be received using some predetermined elements among the plurality of elements;
determining whether a section of interest of an object is included in a weak resolution region determined by the setting; and
generating an ultrasound image of the section of interest according to a beamforming method corresponding to focusing of the transmitted ultrasound by transmitting the ultrasound and receiving the echo ultrasound in accordance with the setting when the section of interest is included in the weak resolution region.

2. The method according to claim 1, wherein the determining of whether the section of interest is included in the weak resolution region includes determining whether results of reception of the echo ultrasound using all of the plurality of elements and using some of the plurality of elements are the same.

3. The method according to claim 1, wherein, in the generating of the ultrasound image, when the ultrasound is focused, a plurality of echo ultrasounds corresponding to a plurality of ultrasounds transmitted along a plurality of scanlines are received, and the ultrasound image of the section of interest is generated based on a coherent sum of at least two echo ultrasounds that include information on the same position among the plurality of echo ultrasounds.

4. The method according to claim 3, wherein the generating of the ultrasound image includes transmitting the plurality of ultrasounds to a plurality of focal points inside the object along the plurality of scanlines using all of the plurality of elements of the 2D array probe.

5. The method according to claim 3, wherein the generating of the ultrasound image includes receiving the plurality of echo ultrasounds that include information on an inside of the object in which the plurality of scanlines are positioned using some elements of the 2D array probe.

6. The method according to claim 3, wherein, in the generating of the ultrasound image, each echo signal corresponding to each scanline is generated by performing a coherent sum of at least two echo ultrasounds that include information on the same position inside the object among the plurality of echo ultrasounds, and the ultrasound image of the section of interest is generated based on each of the echo signal.

7. The method according to claim 1, wherein, in the generating of the ultrasound image, when the ultrasound is not focused, a plurality of echo ultrasounds generated by a plurality of plane waves having different propagating directions are received, and the ultrasound image of the section of interest is generated based on a coherent sum of at least two echo ultrasounds that include information on the same position among the plurality of echo ultrasounds.

8. The method according to claim 7, wherein, in the generating of the ultrasound image, a plurality of plane wave ultrasounds having different propagating directions are transmitted to the object using all of the plurality of elements of the 2D array probe.

9. The method according to claim 7, wherein, in the generating of the ultrasound image, the plurality of plane wave echo ultrasounds generated from an inside of the object by the plurality of ultrasounds are received using some elements of the 2D array probe.

10. The method according to claim 7, wherein, in the generating of the ultrasound image, an echo signal of the object is generated by performing a coherent sum of at least two echo ultrasounds that include information on the same position among the plurality of plane wave echo ultrasounds, and the ultrasound image of the section of interest is generated based on the echo signal.

11. The method according to claim 1, wherein the setting of the 2D array probe includes setting elements arranged in different diagonal directions among the plurality of elements to receive the echo ultrasound.

12. An ultrasonic imaging apparatus, comprising:
a control unit configured to set an ultrasound to be transmitted using all of a plurality of elements of a 2D array probe and an echo ultrasound to be received using some predetermined elements among the plurality of elements;
a computing unit configured to determine whether a section of interest of an object is included in a weak resolution region determined by the setting;
a 2D array probe configured to transmit the ultrasound and receive the echo ultrasound according to the setting;
a beamformer configured to generate an echo signal by beamforming according to a beamforming method corresponding to focusing of the echo ultrasound when the section of interest is not included in the weak resolution region; and
an image processing unit configured to generate an ultrasound image of the section of interest of the object based on the echo signal.

13. The apparatus according to claim 12, wherein the computing unit determines whether the section of interest is included in the weak resolution region in which results of reception of the echo ultrasound using all of the plurality of elements and using some of the plurality of elements are not the same.

14. The apparatus according to claim 12, wherein the beamformer includes a retrospective transmit beamformer that generates the echo signal by performing a coherent sum of at least two echo ultrasounds that include information on the same position among the plurality of echo ultrasounds corresponding to the plurality of ultrasounds transmitted along a plurality of scanlines when the ultrasound is focused and transmitted.

15. The apparatus according to claim 14, wherein the control unit controls the 2D array probe such that the plurality of ultrasounds are radiated onto a plurality of focal points inside the object along the plurality of scanlines using all of the plurality of elements.

16. The apparatus according to claim 14, wherein the control unit controls the 2D array probe such that the plurality of echo ultrasounds that include information on an inside of the object in which the plurality of scanlines are positioned are received using some elements.

17. The apparatus according to claim 14, wherein the retrospective transmit beamformer generates each echo signal corresponding to each scanline by performing a coherent sum of at least two echo ultrasounds that include information on the same position inside the object among the plurality of echo ultrasounds.

18. The apparatus according to claim 12, wherein the beamformer includes a coherent angular compounding beamformer that generates the echo signal by performing a coherent sum of at least two echo ultrasounds that include information on the same position among the plurality of echo ultrasounds generated by a plurality of plane waves having different propagating directions when the ultrasound is transmitted without focusing.

19. The apparatus according to claim 18, wherein the control unit controls the 2D array probe such that a plurality of plane wave ultrasounds having different propagating directions are transmitted to the object using all of the plurality of elements.

20. The apparatus according to claim 18, wherein the control unit controls the 2D array probe such that the plurality of plane wave echo ultrasounds generated from an inside of the object by the plurality of ultrasounds are received using some elements.

21. The apparatus according to claim 12, wherein the 2D array probe sets the echo ultrasound to be received using elements that are arranged in different diagonal directions among the plurality of elements.
